(19) United States
(12) Patent Application Publication
(10) Pub. No.: US 2003/0233063 A1
(43) Pub. Date: Dec. 18, 2003

(54) Applicator and application method

(75) Inventor: Hiroshi Nakatani, Tondabayashi-shi (JP)

Correspondence Address: ROTHWELL, FIGG, ERNST & MANBECK, P.C., 1425 K STREET, N.W., SUITE 800, WASHINGTON, DC, 20005, US

(73) Assignee: SANYO CO., LTD., Osaka (JP)

(21) Appl. No.: 10/458,621

(22) Filed: Jun. 11, 2003

(30) Foreign Application Priority Data
Jun. 13, 2002 (JP) 2002-172337

(51) Int. Cl. A61M 35/00

(52) U.S. Cl. 604/2

(57) ABSTRACT

An applicator includes a shank, an application liquid impregnated part disposed on at least one end of the shank, and a film covering the shank and the application liquid impregnated part. The film includes a shank cover region and an impregnated part cover region formed integrally with and separable from the shank cover region. The shank cover region of the film is formed to have a small width capable of being gripped by a user in the state that the shank is covered with the film. Therefore, it is avoidable that the user's hand becomes dirty with application liquid such as medical liquid by separating only the impregnated part cover region and then gripping the shank through the film covering the shank.

Priority is claimed to Japanese Patent Application No. 2002-172337, filed on Jun. 13, 2002, the disclosure of which is incorporated by reference in its entirety.

### BACKGROUND OF THE INVENTION

1. Field of the Invention

The present invention relates to an applicator for applying medical liquid used for medical care such as disinfection, sterilization and medical treatment, makeup and cleaning of machine parts, electronic parts or the like. The invention also relates to an application method.

2. Description of Related Art

The following description sets forth the inventor's knowledge of related art and problems therein and should not be construed as an admission of knowledge in the prior art.

It has been conventional practice to wind absorbent cotton around one end of a shank, impregnate medical liquid such as disinfectant in the absorbent cotton, and apply this to an external wound or an affected part such as throat. It is, however, tedious to wind absorbent cotton and impregnate the medical liquid therein. In order to avoid this, it is proposed that a fiber aggregate such as absorbent cotton is attached to one end of a shank, medical liquid is then impregnated in the fiber aggregate, and the resultant is sealingly packed individually with a gas impervious film. With the use of this applicator, the medical liquid can directly be applied to the affected part by unpacking the package and pinching the shank with fingers.

However, this applicator suffers from the problem that medical liquid is liable to seep from the fiber aggregate due to temperature changes or when pressure is applied from the exterior during post-manufacture storage or transportation. This results in the possibility that the seeped medical liquid adheres on the surface of the shank and the hand becomes dirty when taking out the shank.

In order to solve this problem, it has been proposed to pack only an application liquid impregnated part (Japanese Patent Unexamined Publication Nos. 11-47191 and 2000-237235). However, by packing only the application liquid impregnated part, it is difficult to maintain gastightness for a long period of time, and there is also the possibility that the application liquid leaks through the shank to the exterior.

The description herein of advantages and disadvantages of various features, embodiments, methods, and apparatus disclosed in other publications is in no way intended to limit the present invention. Indeed, certain features of the invention may be capable of overcoming certain disadvantages, while still retaining some or all of the features, embodiments, methods, and apparatus disclosed therein.

### SUMMARY OF THE INVENTION

Preferable embodiments can provide an applicator and the application method, in which application liquid such as medical liquid is previously impregnated in an application liquid impregnated part, and which precludes the possibility that the hand becomes dirty with the application liquid at the time of use.

Another preferable embodiments can provide an applicator and the application method, with which the application of medical liquid or the like can be performed easily and sanitarily.

An applicator of the present invention includes a shank, an application liquid impregnated part provided on at least one end of the shank, and a film covering the shank and application liquid impregnated part. The film includes a shank cover region and an impregnated part cover region formed integrally with and separable from the shank cover region. The shank cover region of the film is formed in such a small width that the shank can be gripped by a user in the state that the shank is covered with the film. The film is preferably a gas impervious film.

According to an application method using this applicator, an application liquid impregnated part is exposed by separating an impregnated part cover region from a shank cover region in the film. Then, application is performed while gripping the shank covered with the film.

It is therefore possible to apply application liquid such as medical liquid easily and sanitarily, without making the hand dirty with the application liquid. In addition, since a certain amount of application liquid is previously impregnated in the application liquid impregnated part, it is possible to prevent such disadvantages that an excess of impregnated application liquid causes drips, and that the amount of application liquid is insufficient.

The term “region covering the application liquid impregnated part” (impregnated part cover region) in the present invention is to include not only a region directly covering the application liquid impregnated part, but also locations covering a part of the shank adjacent to the application liquid impregnated part, as long as they constitute no obstruction to grip of the shank via the film.

Another objects and features of the present invention will be apparent from the following descriptions with reference to the attached drawings.

### BRIEF DESCRIPTION OF THE DRAWINGS

The accompanying figures are provided by way of example, without limiting the broad scope of the invention or various other embodiments, wherein:

FIG. 1 is a front elevation, partially broken away, of an applicator according to one preferred embodiment of the present invention;

FIGS. 2(a) and 2(b) are partially enlarged views of notch parts;

FIG. 3 is a front view of an applicator employing perforated lines as separating means;

FIG. 4 is a sectional view of a gas impervious film employing perforated lines as separating means;

FIGS. 5(a) and 5(b) are explanatory views showing the use of an applicator of the present invention;

FIG. 6 is a perspective view of the applicator as being used;

FIG. 7 is a front elevation of an applicator according to another preferred embodiment of the present invention;

FIGS. 8(a) and 8(b) are explanatory views showing the use of an applicator according to other preferred embodiment;

FIG. 9 is a front elevation of an applicator according to still another preferred embodiment of the present invention; and

FIG. 10 is a front elevation of an applicator according to still yet another preferred embodiment of the present invention.

### DETAILED DESCRIPTION OF THE PREFERRED EMBODIMENTS

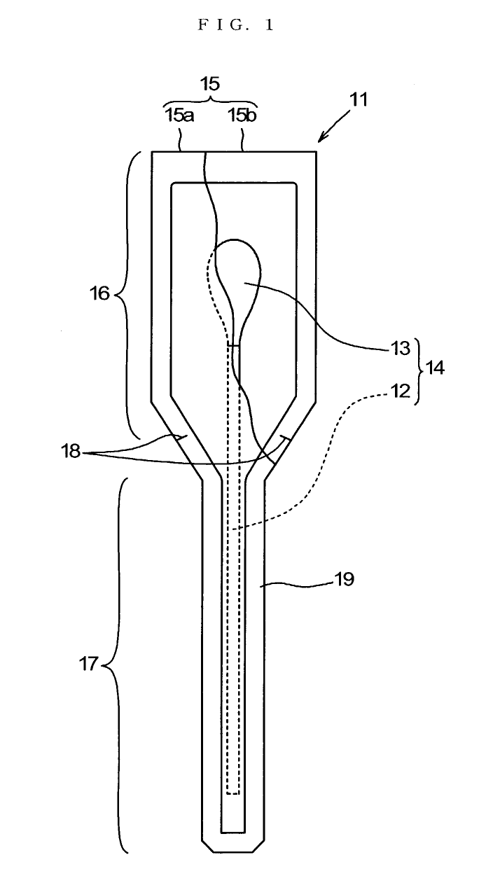

An applicator according to one preferred embodiment of the present invention will be described based on the accompanying drawings. FIG. 1 is a front elevation, partially broken away, of one preferred embodiment of the applicator according to the present invention.

Referring to FIG. 1, an applicator 11 includes a shank 12, an application liquid impregnated part 13 impregnated with application liquid and formed on one end of the shank 12, and a gas impervious film 15 for sealingly packing the shank 12 and the application liquid impregnated part 13. The gas impervious film 15 has, on the peripheral part thereof, a sealing part 19. The sealing part 19 is provided with notch parts 18 (separating means) for separating the impregnated part cover region 16 covering the application liquid impregnated part 13 from the shank cover region 17 covering the shank 12.

The shank 12 can be, for example, a plastic shank, a paper shank, a wood shank and a metal shank. The thickness (outside diameter) of the shank 12 is not particularly limited. Preferably, the shank 12 is strong enough not to be broken during the use and has an easy-to-grip thickness during the use. The length of the shank 12 is also not particularly limited, and it can be set to any easy-to-use length.

As the application liquid impregnated part 13, any material that can impregnate and apply application liquid is usable. There are, for example, plastic foam and fiber aggregate. Alternatively, the part 13 can be one that is obtained by winding a non-woven fabric or woven fabric (e.g., gauze) around the shank 19. Examples of the fiber aggregate include a variety of natural fibers (cotton, silk, wool, etc.), regenerated fibers (rayon, cupra, etc.), and synthetic fibers (polyester fiber, polypropylene fiber, etc.). Most preferred one is cotton fiber such as absorbent cotton. Such fiber is shaped in lump form such as approximately spherical or ellipse, and is, as required, fixed to the shank 12 with adhesive.

The thickness (outer diameter) and length of the application liquid impregnated part 13 are not particularly limited, but preferably has such a size enough to impregnate sufficiently the necessary amount of application liquid. In the following description, for the sake of convenience, the shank 12 having the impregnated part 13 is referred to as a “swab 14” in some cases.

As the gas impervious film 15, any one that can prevent volatilization of the application liquid for a long period of time is usable. For example, there is one that is obtained by laminating a film for thermal bond such as polyethylene film on metallic foil such as aluminum foil. In order to sealingly pack the swab 14 with the gas impervious film 15, gas impervious films 15a and 15b are disposed on upper and lower sides of the applicator 14 such that their respective films for thermal bond are opposed to each other, and the sealing part 19 disposed on the peripheral part is heated and pressed for thermal bond, so that the periphery is subjected to heat sealing, thereby obtaining the applicator 11 that sealingly houses the swab 14 in the gas impervious film 15. Although the swab 14 is sealingly packed with the film for thermal bond in this preferred embodiment, it can be sealed by applying adhesive to the peripheral part, in place of using the film for thermal bond.

Although the shape of the gas impervious film 15 is not particularly limited, its width is preferably changed so as to correspond to the thickness of the shank 12 and the application liquid impregnated part 13, as shown in FIG. 1. In particular, the shank cover region 17 has preferably a narrow width enough to house the shank 12. This facilitates to grip the shank 12 via the gas impervious film 15 at the time of use.

Further, the plastic film, such as the above-mentioned film for thermal bond, in the gas impervious film 15 is preferably a uniaxial oriented film that facilitates unpacking.

The sealing width of the sealing part 19 is not particularly limited and determined in consideration of the volatility of the application liquid to be impregnated in the application liquid impregnated part 13.

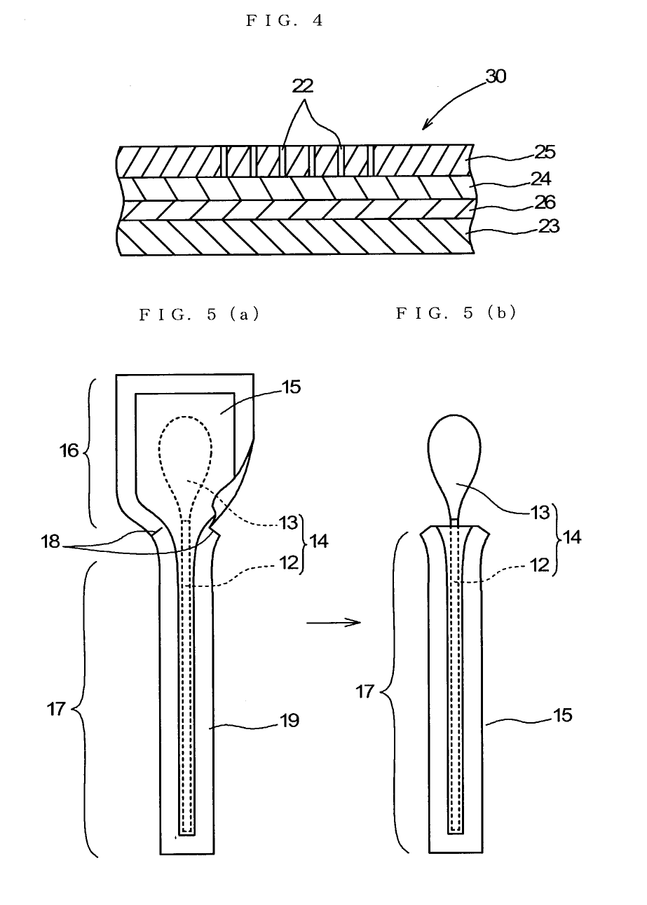

The notch part 18 is provided at a portion of the sealing part 19 which locate between the impregnated part cover region 16 and the shank cover region 17. As shown in FIG. 1, the notch part 18 is obtained by forming a linear slit that extends obliquely from the edge of the sealing part 19 to the impregnated part cover region 16. The notch part 18 can be provided at only one end portion of the sealing part 19. In this case, the gas impervious film 15 cleaves in its width direction because of the uniaxial oriented film. Therefore, the unpacked shank cover region 17 is chamfered at its end part as shown in FIG. 5(b). This prevents that a sharp point erroneously comes into contact with the affected part at the time of application.

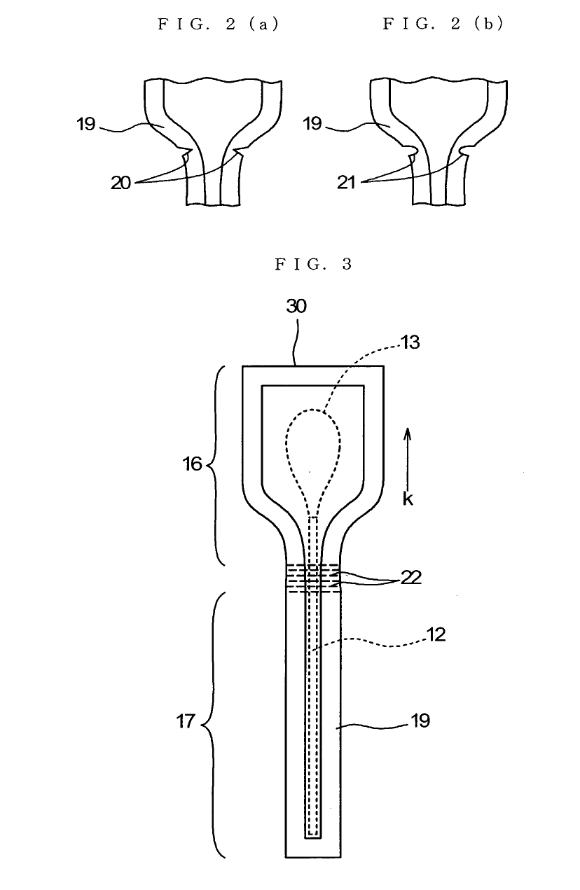

FIGS. 2(a) and 2(b) show another notch parts having different shapes. As shown in FIGS. 2(a) and 2(b), the notch parts 20 and 21 can be cut into a V-shape or a U-shape.

As other means for separating the impregnated part cover region 16 from the shank cover region 17, for example, a plurality of perforated lines 22 can be formed in the width direction of the gas impervious film 30, as shown in FIG. 3. The gas impervious film 30 of FIG. 3 is obtained by laminating and integrating, as shown in FIG. 4, a film for thermal bond 24 and a heat resistance film 25 in this order on the surface of a gas impervious film 26, such as a metallic foil, the underside of which is laminated with a film for thermal bond 23. Then, the plurality of perforated lines 22 are formed in the heat resistance film 25 in the surface of the gas impervious film 30. By drawing the impregnated part cover region 16 at the time of unpacking (the direction of drawing is indicated by arrow “k” in FIG. 3), the gas impervious film 15 cleaves at the region where the perforated lines 22 are formed, and the application liquid impregnated part 13 is exposed. Also in this case, the perforated lines 22 are preferably formed in trapezoid such that the end part of the shank cover region 17 separated from the impregnated part cover region 16 is shaped so as to be chamfered. If it is difficult to do so, linear perforated lines 22 are preferably formed away from the application liquid impregnated part 13, as shown in FIG. 3.

Application liquid to be impregnated in the application liquid impregnated part 13 is properly selected depending on the use and purpose. For example, the application liquid can be medical liquids, makeup liquids, or cleaning liquids of machine parts or electronic parts, etc. It is also possible to impregnate organic solvents, water or other cleaning liquids for removing contamination in human body and clothing.

Examples of the medical liquids include medical liquids used for disinfection, sterilization or medical treatment. There are for example disinfectants such as acrinol, alkyldiaminoethylglycine HCl, ethanol, hydrogen peroxide, glutaral, cresol, chlorhexidine gluconate, sodium hypochlorite, benzalkonium chloride, benzethonium chloride, povidone-iodine (Isodine Solution, etc.), mercurochrome and iodine tincture. As long as these medical liquids are liquid, they can directly be impregnated in the application liquid impregnated part 13. Alternatively, they can be dissolved in a solvent such as water and then impregnated in the application liquid impregnated part 13.

Examples of the makeup liquids include plant extracts such as aloe extract, collagen, lotion, baby oil, baby lotion, mugwort extract, luffa extract and rice sugar extract, and various aromatics. Besides, nail-polish remover and cleansing liquid can be used. As long as these makeup liquids are liquid, they can directly be impregnated in the application liquid impregnated part 13. Alternatively, they can be dissolved in a solvent such as water and then impregnated in the application liquid impregnated part 13.

Examples of the cleaning liquids include organic solvents such as acetone and alcohols, and machine oils.

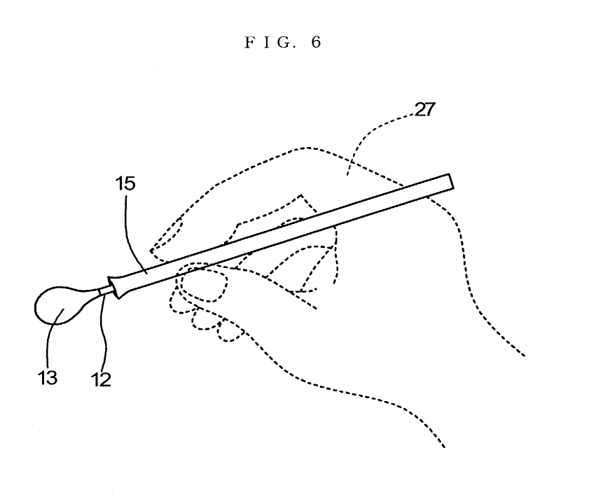

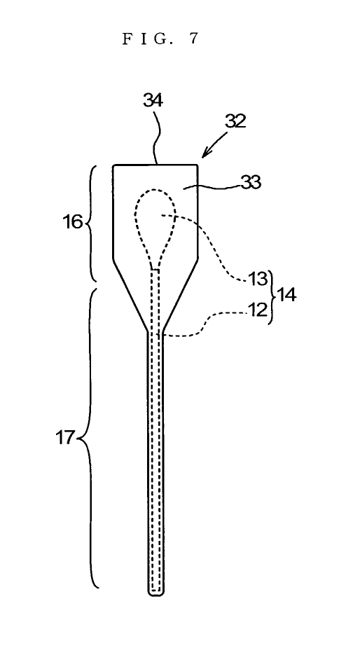

The use of the applicator 11 will be explained with reference to FIGS. 5 and 6 as an example. As shown in FIG. 5(a), the gas impervious film 15 is drawn along the notch parts 18 and the impregnated part cover region 16 is separated and removed from the shank cover region 17, thereby exposing the application liquid impregnated part 13. Then, as shown in FIG. 6, in the state that the shank 12 is covered with the gas impervious film 15 of the shank cover region 17, application is performed while gripping the shank 12 by a hand 27. This manner avoids that the hand 27 becomes dirty during the use, even if during the time that the applicator is stored or transported, the application liquid leaks from the application liquid impregnated part 13 and adheres on the surface of the shank 12.

Further, the remaining end part of the impervious film 15 has a chamfered shape that slants downwardly to its edge part. This prevents that a sharp point of the gas impervious film 15 erroneously comes into contact with the affected part at the time of application. Note that if the film edge part of the shank cover region 17 is sufficiently apart from the application liquid impregnated part 13, such a chamfered shape is not necessarily required. In the case of the V-shape and U-shape notch parts 20 and 21 shown in FIGS. 2(a) and 2(b), respectively, the edge part of the gas impervious film 15 so separated is always chamfered and therefore preferable.

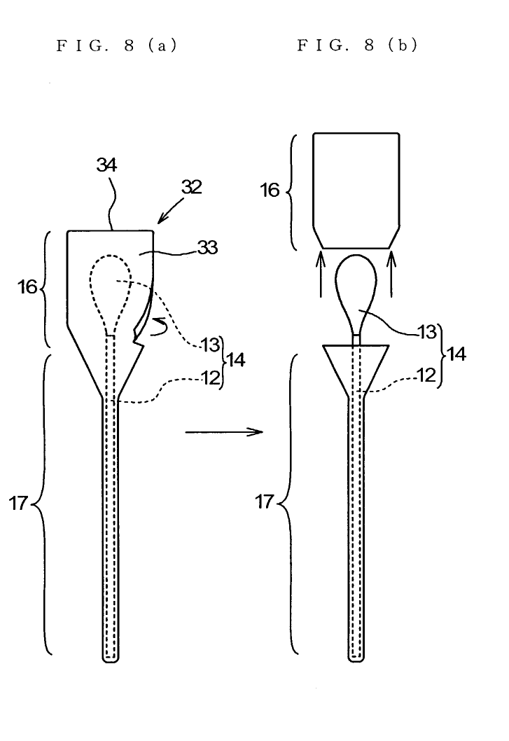

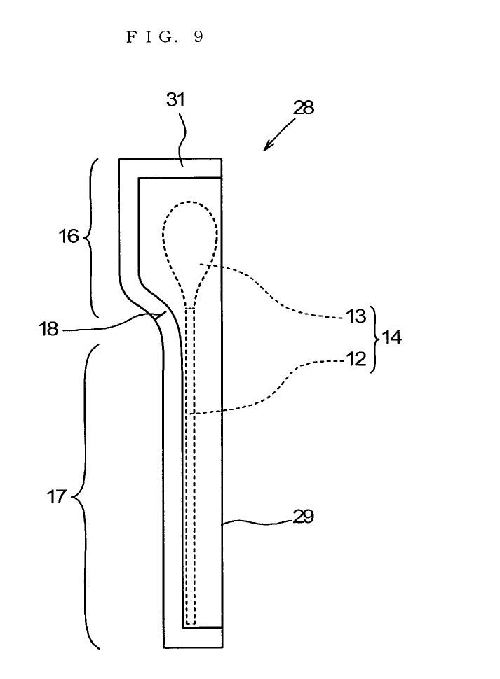

Another preferred embodiment of the present invention is shown in FIG. 7. An applicator 32 is obtained by wrapping a swab 14 in a film for thermal bond 33 and performing heat sealing at the same time that a peripheral part 34 is cut with a heated knife, etc. Since the applicator 32 is obtained only by wrapping the swab 14 in the film for thermal bond 33, this is suitable particularly when the application liquid impregnated in the application liquid impregnated part 13 is low volatile liquid such as oil. It is preferable that the film for thermal bond 33 is made of a uniaxial oriented film and the direction of its orientation is orthogonal to the shank 12. Thereby, at the time of use, the vicinity of the boundary between the impregnated part cover region 16 and the shank cover region 17 can easily be cut away by hand to remove the impregnated part cover region 16 as shown in FIGS. 8(a) and 8(b). If required, the film for thermal bond 33 can be provided with a sealing part for separating the impregnated part cover region 16. Alternatively, it can be separated with the use of scissors, etc. Otherwise, this preferred embodiment is identical with the foregoing embodiment. Accordingly, the same reference numerals are allotted to the corresponding portions, and the descriptions are omitted here.

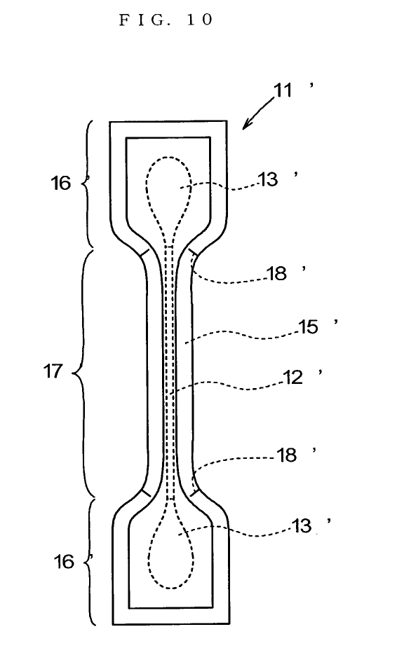

Still another preferred embodiment of the present invention is shown in FIG. 9. This applicator 28 is obtained by folding a gas impervious film 29 sandwiching an applicator body 14 therebetween and then sealing an overlapped peripheral part 31 (sealing part) by thermal bond or the like. Otherwise, this preferred embodiment is identical with the foregoing embodiment. Accordingly, the same reference numerals are allotted to the corresponding portions, and the descriptions are omitted here.

In the foregoing preferred embodiments, the application liquid impregnated part 13 is formed at one end of the shank 12. Alternatively, application liquid impregnated parts 13′ and 13′ can be formed on both ends of a shank 12′, as shown in FIG. 10. In this case, a narrow shank cover region 17′ is formed in a middle part of a gas impervious film 15′, regions 16′ and 16′ covering the application liquid impregnated parts 13′ are formed on both ends of the shank 12′, and notch parts 18′ are formed on the boundaries between the region 17′ and both of the regions 16′, 16′. Otherwise, the construction is identical with the foregoing preferred embodiments.

As required, the shank 12 (12′) can be adhered on the internal surface of the shank cover region 17 (17′) of the gas impervious film.

According to the present invention, an application liquid such as medical liquid is previously impregnated in the application liquid impregnated part and sealingly packed with the gas impervious film. It is, therefore, convenient to use the applicator immediately after unpacking the gas impervious film. In addition, it is avoidable that the user's hand becomes dirty with application liquid such as medical liquid by separating only the region covering the application liquid impregnated part and then gripping the shank via the film covering the shank.

While illustrative embodiments of the present invention have been described herein, the present invention is not limited to the various preferred embodiments described herein, but includes any and all embodiments having modifications, omissions, combinations (e.g., of aspects across various embodiments), adaptations and/or alterations as would be appreciated by those in the art based on the present disclosure. The limitations in the claims are to be interpreted broadly based the language employed in the claims and not limited to examples described in the present specification or during the prosecution of the application, which examples are to be construed as non-exclusive. For example, in the present disclosure, the term “preferably” is non-exclusive and means “preferably, but not limited to.” Means-plus-function or step-plus-function limitations will only be employed where for a specific claim limitation all of the following conditions are present in that limitation: a) “means for” or “step for” is expressly recited; b) a corresponding function is expressly recited; and c) structure, material or acts that support that structure are not recited.

What is claimed is:

1. An applicator, comprising:
a shank;
an application liquid impregnated part provided on at least one end of said shank; and
a film covering said shank and said application liquid impregnated part,
wherein said film includes a shank cover region covering said shank and an impregnated part cover region covering said application liquid impregnated part, said impregnated part cover region being formed integrally with and separable from said shank cover region, and wherein said shank cover region of said film is formed to have a small width capable of being gripped by a user in the state that said shank is covered with said film.

2. The applicator according to claim 1, wherein said film is a gas impervious film.

3. The applicator according to claim 2, wherein said gas impervious film is a film obtained by laminating a film for thermal bond on a metallic foil.

4. The applicator according to claim 2, wherein said gas impervious film is provided with a sealing part for sealingly packing said impregnated part cover region and said shank cover region.

5. The applicator according to claim 4, wherein said sealing part is provided with a separating portion to separate said impregnated part cover region from said shank cover region.

6. The applicator according to claim 5, wherein said separating portion is a notch part.

7. The applicator according to claim 1, wherein said film is subjected to heat sealing at the same time it is cut.

8. The applicator according to claim 1, wherein said application liquid impregnated part is a foam body or fiber aggregate having liquid absorbing property.

9. The applicator according to claim 1, wherein said application liquid is liquid selected from the group consisting of medical liquid, makeup liquid and cleaning liquid.

10. An application method using the applicator according to claim 1, the application method, comprising:
exposing said application liquid impregnated part by separating said impregnated part cover region from said shank cover region; and
performing application while gripping said shank covered with said film.
